(19) United States
(12) Patent Application Publication
(10) Pub. No.: US 2020/0121870 A1
(43) Pub. Date: Apr. 23, 2020

(54) LOW-COST SINGLE USE POWDER INHALER

(71) Applicant: MYLAN INC, CANONSBURG, PA (US)

(72) Inventor: MICHAEL JOHN HOLROYD, DUBLIN (IE)

(73) Assignee: MYLAN INC, CANONSBURG, PA (US)

(21) Appl. No.: 16/500,837

(22) PCT Filed: Apr. 3, 2018
(86) PCT No.: PCT/US18/25806

§ 371 Date: Oct. 4, 2019

Related U.S. Application Data
(60) Provisional application No. 62/482,259, filed on Apr. 6, 2017.

(51) Int. Cl. A61M 15/00

(52) U.S. Cl. CPC A61M 15/0043; A61M 15/0021; A61M 15/0051; A61M 2205/273; A61M 2202/064

(57) ABSTRACT

The present invention relates to a low-cost single-use disposable breath actuated dry powder inhaler, its method of use, and method of manufacture. The inhaler comprises a bottom layer and a top layer. The inhaler comprises a medicament compartment and an air passageway. Furthermore, the medicament compartment is sealed by a flexible pull tab that is easily removed before use, and the air passageway comprises a mouthpiece port, a Venturi section, and an air inlet port.

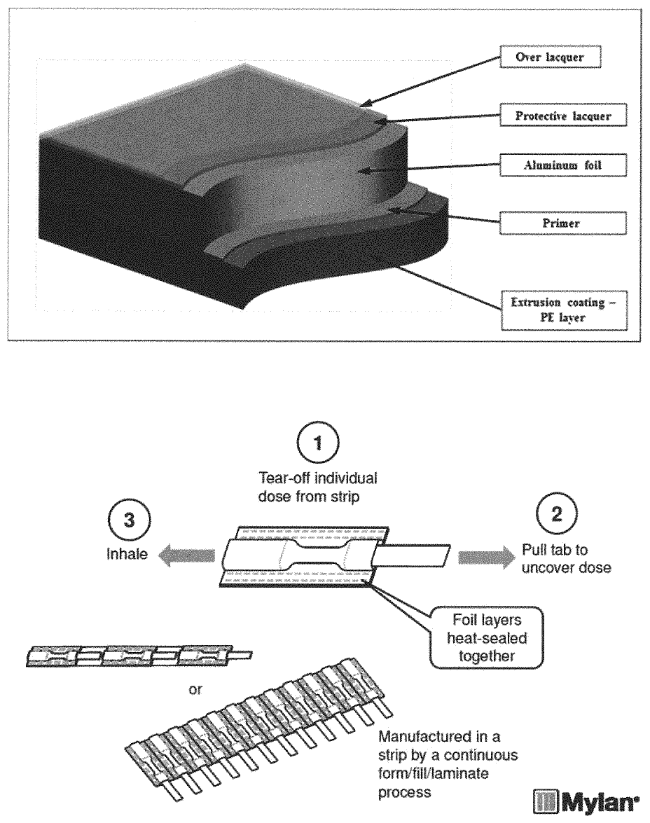

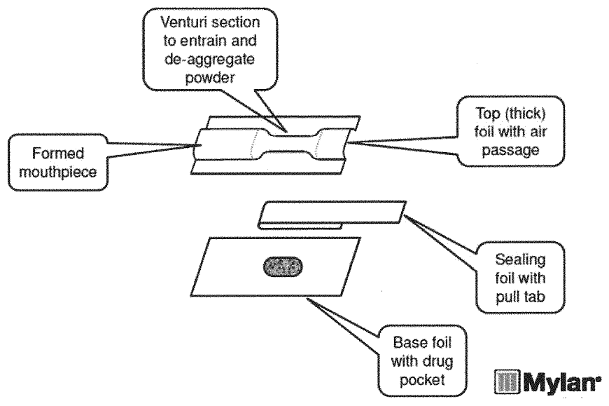

### FIELD OF THE INVENTION

The present invention relates to a low-cost single-use breath actuated dry powder inhaler wherein the inhaler comprises a bottom layer and a top layer with both layers sealed together to define a medicament compartment and an air passageway. Furthermore, the medicament compartment is sealed by a pull tab that is easily removed before use. Additionally, the air passageway comprises a mouthpiece port, a Venturi section, and an air inlet port.

### BACKGROUND OF THE INVENTION

Medicinal aerosol therapy, in which an active constituent is absorbed by inhalation through the lungs, plays an important role in the treatment of many lung diseases. In order to administer the medicaments nebulisers, metering aerosols, or dry powder inhalers are often used. In the field of powder inhalers single-dose appliances and multi-dose appliances are known. Dry powder inhalers basically comprise a dose supply region for the powdered medicament, a mouthpiece section and a de-aggregation area to de-aggregate the powdered medicament.

Dry powder inhalation devices are known which comprise a body or housing within which a blister or pocket-containing medicament pack is located. Such medicament packs may comprise a base sheet in which the blister or pocket is formed and a lid sheet which covers the pocket/blister. Typically, the drug is accessed from the blister or pocket by peeling back or rupturing a section of the lid sheet to expose the medicament within the blister/pocket. Although a variety of inhalers are known for the administration of dry powder medicaments, each of these inhalers suffers certain drawbacks

U.S. Pat. No. 8,424,518 describes a dry powder medicament cartridge for an inhaler, comprising a cartridge top and a cartridge bottom which are moveable relative to one another by a translational motion; at least one inlet port to allow flow into the enclosure, and at least one dispensing port to allow flow out of the enclosure. The need for an additional inhalation device that may be reusable and the incorporation of a moving parts adds to the cost and complexity of the inhalation device, both in terms of use by the patient and device manufacturability.

EP1726324 describes a dry powder medicament blister for use in a reusable device requiring a piercing mechanism. The medicament blister has an upper layer, a middle layer, and a bottom layer having at least one receptacle, in which medicament is disposed. The upper layer is punctured to provide first and second openings to allow airflow to enter and exit through the upper layer of the medicament container. The incorporation of a piercing mechanism in an additional device adds to the cost and complexity of the inhalation device, both in terms of use by the patient and device manufacturability.

US20070221218A1 describes multi-dose dry powder drug containment packages with tab members and metered dose sealed drug compartments that can be used in an inhaler having a flow chamber with a hook member that opens the drug compartments and/or a portion that flexes to vibrate the dry powder to facilitate fluidic active dispersion for inhalation. In this prior art example, the drug containment system must be kept for multiple uses. Additionally, the device is made up of multiple components and has a relatively complex structure.

U.S. Pat. Nos. 6,915,802 and 7,032,593 both describe single-use disposable inhalation devices that require sliding action of the components. U.S. Pat. No. 6,915,802 comprises first body portion having a pocket and a second body portion having an air path. The pocket is sealed to a foil cover which connects to the second body portion. The second body portion is movable over the first body portion to break the seal and bring the pocket into communication with the air path. U.S. Pat. No. 7,032,593 describes a device having a first casing portion and a second casing portion. The casing portions are coupled such that they may slide with respect to each other. The casing contains a powder chamber, and has at least one ventilation opening to allow air to enter into the casing. The casing also includes a powder outlet opening. The powder chamber is configured such that sliding the first casing portion relative to the second casing portion causes the powder chamber to move from a closed position to an opened position. In the opened position, powder from the powder chamber can exit the device through the powder outlet opening. Although these devices are light and compact, allowing for convenient storage and portability by a patient, both devices require movable parts and are relatively more complex to manufacture than the present invention.

EP2526990 describes an inhaler comprising a first body member and a second body member. The first body member and the second body member fitting closely together, preferably hinged together, to form an inhaler body and an outlet. The first body member including a medicament chamber containing a unit dose of powdered medicament and the first body member having affixed to it a foil that closes the medicament chamber and which extends outwardly of the inhaler body such that, in use, it can be grasped by a user and withdrawn, fully or partially, from the inhaler body releasing the powdered medicament from the medicament chamber. The second body member including a medicament collection well in which the medicament is collected when released, and the medicament collection well is part of an airway for inhalation of the medicament. Although, the inhaler may be disposable and suitable for single-use, the inhaler requires multiple movable parts hinged together which can break and is relatively more complex to manufacture than the present invention.

De-aggregation is often necessary to cause powder particles to independently flow into and from a dose supply region and allows adsorption of the medicament molecules contained in the powder by the alveoli. That is, the tendency of powder particles to stick to one another will negatively impact both the flow of powder required for proper operation and ultimate absorption by the patient's tissues. Thus, de-aggregation is typically an important consideration. To facilitate flow of the powdered medicine, the active pharmaceutical ingredient (“API”) particles are often carried in soft spherical pellets or on the surface of so called carriers. The pellets and/or carriers may or may not need to be de-aggregated into smaller parts inside the inhaler. Thus, the term “independently” includes some level of aggregation under circumstances where aggregation of API with, for example, carriers is a design feature of a particular powder. De-aggregation therefore contemplates reducing aggregation to a level in line with the design parameters of a particular powder formulation.

De-aggregation under breath action can be effectuated in many ways, e.g. by passing the powder particles through air circulation chambers as disclosed in U.S. Pat. No. 7,617,822, but is realized in the present invention by use of a Venturi section. Compared to other de-aggregation principles, a Venturi has the advantage of a de-aggregating particles, while further providing ease of manufacture and low-cost.

U.S. Pat. No. 5,239,993 describes a breath actuated multi-use dry powder medicament inhalator comprising a primary air passageway with a Venturi section and a secondary air passageway which includes a compound de-aggregating swirl chamber that is connected to the Venturi of the primary air passageway. In the known inhaler, a storage chamber is formed in the inhalator apparatus housing for storing the compound to be inhaled. A secondary air passageway is also provided in the inhalator apparatus housing having an air introduction portion at one end thereof adjacent the air inlet portion of the primary air passageway, and the other end thereof is positioned to fluidly communicate with the Venturi portion of the primary air passageway. An enlarged compound swirl chamber is provided in the secondary air passageway between the air introduction portion and the other end thereof. Finally, a dose introduction means is provided within the housing for transporting a predetermined portion of the powder compound from the storage chamber to the compound swirl chamber of the secondary air passageway. Although efficient, the disadvantage of this inhaler is that it needs to be kept by the patient for multiple uses, is relatively bulky, and is relatively costly to manufacture compared to the present invention.

Thus, the inventors have identified the need for a low-cost device for inhalation of dry powder medicaments. Such a low-cost inhaler has medication pre-stored therein that requires minimal steps by a user prior to use. The inhaler comes in a size and shape for portability and convenient storage by a user, and is designed to avoid spillage or other events leading to inaccurate administration of medication. It is also advantageous that such a device administer an individual dose of medicament in a disposable unit.

### BRIEF SUMMARY OF THE INVENTION

The present invention relates to a single-use disposable inhalation device, method of use, and method of continuous manufacturing of such single-use disposable inhalation devices. In one aspect of the present invention, a single-use disposable inhaler for inhaling dry powder medicament is provided. The inhaler comprises a top layer and a bottom layer sealed together and comprising a mouthpiece port connected via an air passageway to an air inlet port, a medicament compartment sealed by a flexible foil with a pull tab and wherein the medicament compartment is adjacent to the air inlet port and the pull tab extends out of the air inlet port or the mouthpiece port.

Another aspect of the present invention is the simplicity in its method of use. To use the inhaler, a patient removes the pull tab from the inhaler releasing the medicament stored in the medicament compartment, and inhales through the mouthpiece port drawing the medicament into the lungs.

Another aspect of the present invention is its method of continuous manufacture. To manufacture a continuous sheet of inhalers, wherein the top layer and the bottom layer are provided in continuous sheets; the mouthpiece port, air passageway comprising a Venturi section, the medicament compartment and air inlet port are continuously formed in one or both of the top layer and the bottom layer; a machine incorporating a filling system fills the medicament compartment; the medicament compartment is sealed by a flexible foil with a pull tab; and the top layer and the bottom layer are sealed together.

One feature of the present invention is its compact and convenient size and shape. Another advantageous feature of the present invention is the accuracy of medicament dosage delivered. Because only one dosage of medication is present in the inhaler during each use, the possibility of overdose is eliminated, and the medicament need not be metered prior to delivery. A patient may simply inhale all medicament present in the device.

The present invention also possesses the advantage that the medicament is stored inside the inhaler. No capsules or other storage units need be carried other than the inhaler itself. Additionally, the medicament is sealed securely inside the inhaler until only a moment prior to use, and thus the chances of spillage are greatly reduced. Because the present invention operates only under inhalation power of the patient, the inhaler carries the additional advantage that no accessory device, such as a compressed air cylinder or other propellant, needs to be used in conjunction with the present invention.

Another advantage of the present invention is that the medicament has been stored in a sealed, dry environment until just prior to administration. Also, during inhalation, the medicament is subjected to mixing by a Venturi section contained in the device. The Venturi section helps to ensure that the medicament exiting the inhaler and entering the patient's respiratory system is in the form of a fine dry powder, facilitating medicament deposition in the lungs. Inhalation of finer powders is typically more comfortable for the patient.

An important advantage of the present invention is that it is disposable. A patient can simply use the inhaler, and dispose of it. Other steps such as cleaning, refilling and permanently storing are unnecessary.

The present invention also has the advantage of ease of use. There are only two steps required to use the present invention. The patient simply removes the pull tab and inhales the medicament through the mouthpiece port.

Another advantage of the present invention is that the inhalers may be manufactured continuously. The manufacturing process helps lower the cost of each inhaler and provides an additional means of sale by marketing multiple inhalers in continuous strips.

### DETAILED DESCRIPTION OF THE INVENTION

The present invention provides an improved device, method of use, and method of manufacture for dry powder low-cost single-use disposable inhalers. As will be described in more detail below, a device of the present invention is an inhaler having a compact and convenient shape. This inhaler contains a single dose of dry powder medicament, and may be disposed of following use by a patient. The inhaler includes features for allowing air to pass into and through the device as a patient inhales. In proper use, air will exit the inhaler carrying a full dose of medicament in the form of a fine, dry, de-aggregated powder, with little risk of spillage. Following use, the patient may dispose of the inhaler.

In an exemplary embodiment, the dry powder inhaler for administration of a unit dose of a medicament comprises a top layer comprising a mouthpiece port connected via an air passageway to an air inlet port, a bottom layer comprising a medicament compartment sealed by a flexible pull tab; wherein the top layer and bottom layer are sealed together and the medicament compartment is adjacent to the air inlet port and the pull tab extends out of the air inlet port. Alternatively, any or all of the mouthpiece port, air passageway or air inlet port could be formed in the bottom layer while the medicament compartment could be formed in the top layer. Further, any of these structures may comprise portions defined in both the bottom and top layers and fully realized once the layers are sealed together. In addition, the flexible pull tab may extend out of the mouthpiece port.

Furthermore, the inhaler includes an air inlet port to allow a stream of ambient air to enter as the user draws air from the inhaler through the mouthpiece port. The air passageway connecting the air inlet port to the mouthpiece port may include a Venturi section. The purpose of the Venturi section is to de-aggregate the dry powder medicament. Adjacent to the air inlet port is a medicament compartment. A dry powder medicament is stored in the medicament compartment, and the medicament compartment is sealed by a flexible pull tab extending out of the air inlet port or mouthpiece port when the top layer and bottom layer are sealed together.

Supplemental to of in place of the Venturi section, a swirl chamber may be formed between the medicament compartment and the mouthpiece port. The action of the swirl chamber may be effectuated by the inclusion of a secondary air passageway having an air introduction portion separate from the air inlet portion of the primary air passageway with the other end thereof positioned to fluidly communicate with the swirl chamber and to enter at an angle oblique to the primary airflow carrying the medicament powder.

A number of materials for the top and bottom layer may be appropriate for achieving the goals of the presently described inhaler. One preferred embodiment of the present invention utilizes a foil material; more particularly, a laminated, extrusion-coated aluminum foil. One side is pre-lacquered and over-lacquered whilst the other side has a primer and polyethylene coating. The polyethylene coating provides the sealing interface for joining the top and bottom layers together. The thickness of the aluminium layer is chosen to give the appropriate mechanical and barrier properties. Thickness needs to be sufficient to provide sufficient rigidity to the inhaler but should be limited to ease formation of the various structures and to keep material costs down.

A preferred embodiment of the flexible pull tab may also comprise a laminated and/or extrusion-coated aluminium foil. The thickness of the aluminium foil used for the pull tab would likely be much less than for the top and bottom layers, given the greater need for flexibility when it comes to the pull tab.

The method of using the present invention utilizes an inhaler to administer a single dose of dry powder medicament. A user utilizes the method of the present invention by removing the pull tab from the inhaler. The fine powder medicament contained therein is exposed to air flowing through the inhaler and, thus, inhaled by the user. The user may dispose of the inhaler after use.

When provided to a user, such as a patient, the inhaler will be provided with a proper dosage of medicament in the medicament compartment. As noted, the inhaler medicament compartment is sealed, preventing moisture in the ambient air from reaching the dry powder medicament housed therein, reducing clumping and thereby facilitating inhalation when a dose is administered. Multiple inhalers may be provided in a continuous strip. Before administration, the user may preferably tear one inhaler off the continuous strip along a perforated line in-between consecutive inhalers.

When a patient is prepared to administer a dose of medicament, the patient then preferably holds the inhaler substantially parallel to the ground and gently removes the pull tab protruding from the air inlet or mouthpiece port. Removing the pull tab serves to remove the seal from the medicament compartment, and to expose the medicament to the air passageway. Preferably, the pull tab is completely removed from the inhaler by the patient prior to inhalation.

The inhaler is now ready for use in administering a dosage of medicament. The patient's lips form an airtight seal about the mouthpiece port. At this time, a relatively forceful inhalation action by the patient will draw air in through the air inlet port. The incoming air is forced over the dosage of medicament contained in the medicament chamber. Preferably, the powder medicament used will consist of independent particles. Such particles are highly susceptible to aerolization, i.e. they readily mix with ambient air. The stream of air passing through the air passageway causes the fine particles of medicament to become airborne. As the airborne medicament passes through the Venturi section, any remaining clumps of medicament are de-aggregated. Breaking up the medicament is another way of recognizing the relative independence of particles, one from another appropriate for effective respiration into the patient's respiratory tract, coating of respiratory tract tissues and ultimate absorption thereof, where appropriate. De-aggregation also makes inhalation more comfortable for the patient.

Preferably, a full dose of medicament will be administered by a single inhalation action by a patient. However, depending on a size of the dose, a patient's lung capacity generally, and a particular inhalation specifically, further inhalations may be necessary. The inhaler may be in part or completely formed of a translucent or transparent material, revealing to a user whether a full dosage of medicament has been properly administered. Alternatively, a patient may have to learn through experience how to best self-administer a full dosage. Preferably, the inhaler is disposed of after use.

The method of manufacture of the present invention uses a continuous manufacturing process to produce low-cost single-use disposable dry powder inhalers. Two strips of material are provided, one strip designated the top layer and the other strip designated the bottom layer. The mouthpiece port, air passageway comprising a Venturi section, medicament compartment and air inlet port are continuously formed in the top and bottom layers. A machine incorporating a filling system then continuously fills the medicament compartments. Next, the medicament compartment is sealed by a flexible pull tab. Finally, the top layer and the bottom layer are sealed together. A perforated line is placed in-between each consecutive inhaler allowing easy separation of an inhaler from the sheet of inhalers. Further, various cutting and forming steps may be applied to the strip of inhalers. One such cutting and forming steps includes the formation of a comfortable shape whereby the user engages the inhaler with their lips.

What is claimed is:

1. A dry powder inhaler for administration of a unit dose of a medicament comprising a top layer,
a bottom layer,
a flexible pull tab, and
powdered medicament,
wherein the top layer and the bottom layer are sealed together and define structures comprising a mouthpiece port connected via an air passageway to an air inlet port, a medicament compartment adjacent to the air inlet port, the air passageway passing over the medicament compartment, and the pull tab coveting the medicament compartment and retaining the powdered medicament therein.

2. An inhaler according to claim 1, wherein the air passageway comprises a Venturi section.

3. An inhaler according to claim 2, wherein the top layer is comprised of foil and the bottom layer is comprised of foil.

4. The inhaler according to claim 1, wherein each of the top and bottom layers comprise laminated, extrusion-coated aluminum foil.

5. A method of operating an inhaler according to claim 1, comprising
removing the pull tab from the inhaler, and
inhaling through the mouthpiece port.

6. A method of manufacturing a continuous sheet of inhalers according to claim 1, wherein
the top layer and the bottom layer are provided in continuous sheets;
the mouthpiece port, the air passageway comprising a Venturi section, and the air inlet port are continuously formed in the top layer;
the medicament compartment is continuously formed in the bottom layer;
a machine incorporating a filling system continuously fills the medicament compartments;
the medicament compartments are continuously sealed by a flexible foil with a pull tab; and
the top layer and the bottom layer are continuously sealed together.
